SEQUENCE LISTING (1 sequence)

SEQ ID NO: 1 - protein, 640 aa, Fusarium graminearum
MHLLNGKTAAVALALLNSCNALKVTILADTNRDGKVDNNDINGKSTWTNNRGALILPNIGDTGSRCAKQWGPSVDIQGDESYLDKCNDASDNVQRNPKYLASLKTLPLTTLSATAKGSIIIADKTGASKVRIFVKQSGKWNYVAADHVFTAKELKSGLELGVDARDVRRPQDWNGYAKIQFTVTDGKTKATDAVAVRVAPVLTHHHGQHAQRIFTTGVNEAGVNKVQETFIADILRNVAGAGIKEPVFQFHNQDIWTQDFFEPGYASIPGPNGPVSIRIMIRSAQSSRRSGRDAFHDLRNDQVGAVQHPGDGDSIDSTGNLETIPPYSHNGKSFPVGRTIMGAWDGRAPLMVEFLKAQQVQEPLILDTSWLYVGHVDEFIQFLPSNNKLGWVIMVADPMKGVDLLKKAVKTGHGKVKAVSRPLSADEKKEQLCLPRQTIAEALKFKSFDAINKHSAERIQANLDIIKRETGITDEDIHRVPALFYYTQSNSWLCPGETAEDDSAQPQKAASNSGITMKTSQGGPGFKAKSIVEAATPGKSIQRRVIDPATQVTALYPGSVNGLVMTDTKILAPSPWGPVINKQDIFAAAVSQVYTNAGYNVTYQDDWFSHFKLQGDVHCGSNSWREIPKKWWDSLRVNNY

(19) United States
(12) Patent Application Publication
(10) Pub. No.: US 2021/0120857 A1
(43) Pub. Date: Apr. 29, 2021

(54) USE OF PEPTIDYLARGININE DEIMINASE TO OBTAIN IMPROVED FOOD

(71) Applicant: DSM IP ASSETS B.V., Heerlen (NL)

(72) Inventors: Monica Diana VLASIE, Echt (NL); Helena Maria NAN, Echt (NL); Arjen SEIN, Echt (NL)

(21) Appl. No.: 15/734,102

(22) PCT Filed: Jun. 3, 2019
(86) PCT No.: PCT/EP2019/064272

§ 371 Date: Dec. 1, 2020

(30) Foreign Application Priority Data
Jun. 7, 2018 (EP) 18176426.7

(51) Int. Cl. A23L 27/00; A23C 11/10; A23L 11/00; A23L 11/30; A23L 2/66; A23L 33/17

(52) U.S. Cl. CPC A23L 27/88; A23C 11/103; A23L 11/07; C12Y 305/03015; A23L 2/66; A23L 27/86; A23L 33/17; A23L 11/33

(57) ABSTRACT

The present invention describes a process for modifying sweetness, liquorice, astringency, powdery/chalk, fulness, thickness and/or digestibility and/or protease inhibitory activity of a protein comprising food.

### FIELD

The present invention relates to the food field.

### BACKGROUND

In a world with a growing population there is an increased demand of proteins. To respond to this growing demand of proteins, there is a need to look at wider applications of proteins. In addition, it is desired to look for plant proteins as an alternative for animal proteins, since it is considered that plants are a more sustainable source of proteins than animals. The use of plant proteins in food is still limited in part because of poor digestibility, taste and nutritional value.

Poor digestibility of proteins in diets containing less refined cereals and grain legumes is due to the presence of less digestible protein fractions, high levels of insoluble fibers and/or high concentrations of antinutritional factors present or formed during processing. Dietary antinutritional factors (ANFs) are known to negatively affect the protein digestibility, bioavailability of amino acids and protein quality of foods (G. Sarwar Gilani et al, British Journal of Nutrition (2012), 108, S315-S332)).

The presence of high levels of trypsin inhibitors in soybean and other grain legumes have been shown to cause significant reduction in protein digestibility (up to 50%) in rats and pigs (G. Sarwar Gilani et al, British Journal of Nutrition (2012), 108, S315-S332). On the basis of the residues at their inhibitory sites, these inhibitors are either a lysine—or arginine—type inhibitors. The soybean is the major source of trypsin inhibitors, accounting for approximately 6% of the protein in the defatted soybean meal. There are two major types of trypsin inhibitors—the Kunitz soybean trypsin inhibitor (KSTI) with an arginine at the reactive site and the Bowman-Birk inhibitor (BBI) with a lysine at the inhibitory site.

Moist heat treatment (100° C. for 20 min) of soybean deactivates a part of the inhibitors, however significant original trypsin activity remains. Prolonged heating required to completely destroy the inhibitory activity would significantly reduce the protein digestibility and quality of the soybean products (G. Sarwar Gilani et al, British Journal of Nutrition (2012), 108, S315-S332). Several commercial soya beverages have been reported to contain up to about 70% of the total inhibitory activity. As well, soya based infant formulas were found to contain 28%. Trypsin inhibitors in raw soybeans cause growth inhibition and pancreas enlargement in susceptible animals.

Another important factor in making plant-based protein beverages is their taste and mouthfeel. Vegetable proteins suffer from impaired taste such as bitterness and increased mouthfeel even at low protein content. Also, many plant protein beverages contain very low amounts of proteins making them less nutritious and less tasty foods.

WO2008/000714 discloses a protein arginine deiminase and the use of this enzyme in the preparation of a food product with an increased amount of citrulline.

WO2017/009100 discloses a process to improve the solubility of a plant protein, for instance pea, soy and rice protein, wherein the plant protein is incubated with a peptidyl arginine deiminase. The foam capacity of plant protein such as pea protein was reduced after incubation with peptidyl arginine deiminase.

There is a need in the art to improve the properties of (plant) protein comprising food.

### FIGURES

FIG. 1: The absorbance changes in time (BAEE continuous assay as described in the example 1) directly related to the trypsin activity in the assay. Sample “min PAD” states the trypsin activity in the presence of the inhibitory protein while “plus PAD” states the trypsin activity measured in the presence of the inhibitory protein pretreated with PAD enzyme. The arrow indicates the extent of reduction of trypsin inhibitory activity (TIA) by the action of the enzyme PAD.

FIG. 2: The NH₂ released in time from the RPI in the SYMPHYD digestion model without enzyme treated (open circles) and with PAD enzyme treated (black circles). The arrows indicate the addition points for pepsin at 5 minutes and pancreatin at 80 minutes respectively.

FIG. 3: Sensory panel assessment of soy milks with and without PAD enzyme treatment as described in the example 4. The black bars represent the sensory aspects of the soy drink and the pattern filled bars represent the sensory aspects of soy drink treated with PAD enzyme. The attribute “powdery-chalk-mf” represents the powdery/chalk mouthfeel, “fullness-mf” represents fullness mouthfeel and “thick-mf” represents thickness in the mouth.

FIG. 4: Sensory attributes of a 2% solution of rapeseed protein isolate with and without PAD treatment. ▪: Sensory attributes of rapeseed protein isolate solution untreated with PAD; ♦: sensory attributes of rapeseed protein isolate solution treated with 2 U PAD per L; ▴: sensory attributes of rapeseed protein isolate solution treated with 20 U of PAD per L; ●: sensory attributes of rapeseed protein isolate solution treated with 60 U of PAD per L. Sensory attributes are astringency mouthfeel (I), flavor intensity (II), sweet flavor (III), bitter flavor (IV), liquorice flavor (V), bitter aftertaste (VI), length aftertaste (VII), and astringent aftertaste (VIII).

FIG. 5: Isoelectric focusing (IEF) gel of various vegetable drinks incubated with PAD enzyme: lane 1—pea drink (Bolthouse Farm unsweetened); lane 2—pea drink (Bolthouse Farm unsweetened) incubated with PAD; lane 3—soy drink (Provamel unsweetened); lane 4—soy drink (Provamel unsweetened) incubated with PAD; lane 5—pea drink (Ripple unsweetened); lane 6-pea drink (Ripple unsweetened) incubated with PAD and lane 7 the pl marker.

FIG. 6: PAD enzyme relative stability with varying pH and temperature. The enzyme activity was set to 100% at pH 7 and 4° C. incubation prior activity measurement. In triangles—the enzyme stability at 37° C.; the circles represent enzyme stability at 4° C. as described in the example 7.

### SEQUENCE LISTING

SEQ ID NO: 1 Peptidyl arginine deiminase from Fusarium graminearum

### SUMMARY

The present invention relates to a process for modifying sweetness, liquorice, astringency, powdery/chalk, fulness, thickness and/or digestibility and/or protease inhibitory activity of a protein comprising food.

The invention also relates to the use of a peptidyl arginine deiminase (PAD) for modifying sweetness, liquorice, astringency, powdery/chalk, fulness, thickness and/or digestibility and/or protease inhibitory activity of a protein comprising food.

The invention also relates to a nutritional supplement comprising a peptidyl arginine deiminase (PAD).

### DETAILED DESCRIPTION

Poor digestibility of vegetable proteins is partly due to the high levels of antinutritional factors (ANFs) such as trypsin inhibitors. The mouthfeel of a drink prepared from plant proteins is typically described as being not acceptable. Until the present invention, no acceptable solution is available to overcome these drawbacks of a plant protein drink. This is equally applicable to protein comprising foods in general.

PAD deiminates arginine residues in proteins to citrulline. For the trypsin inhibitors in which arginine occupies the reactive site, PAD action would render the inhibitor inactive towards trypsin. Chymotrypsin protease inhibitors and α-amylase inhibitors contribute as well to lowering food digestibility in a similar manner to trypsin inhibitors. Several of these inhibitors were shown to possess a crucial arginine residue important for the inhibitory function (Gideon M. Polya, Atta-ur-Rahman Ed. Studies in Natural Products Chemistry, vol 29, p 567-641).

About 10% of all proteases inhibitors contain an arginine (R) residue at the reactive, P1 site (309 sequences from 3300 found in the MEROPS database for protease inhibitors). Trypsin inhibitors with “R” at P1 site can be found among all classes of vegetable proteins such as legumes, cereals, nuts and seeds (soy, rapeseed, nuts, wheat, maize, barely, potatoes, rice, oats, tomatoes etc.), however not all inhibitory activities are found equally distributed within different species. For example, about 40% of proteins in potato are protease inhibitors with many different classes of protease inhibitors present (more than 10 different proteases inhibitors identified) with distribution of these inhibitors dependent on the potato variety (Pouvreau L. et al., J. Agric. Food Chem., 2001, vol 49, p 2864-2874).

Deactivation of Kunits soybean trypsin inhibitor was shown before (H. Takahara et al., 1985, The Journal of Biological Chemistry, vol. 260, No. 14, pages 8378-8383) using a mammalian PAD enzyme. However, deactivation of TIA (trypsin inhibitor activity) in a complex protein matrix such as soy flour was never shown. For a benefit in a food product, a PAD activity in a complex food matrix is essential. In addition, mammalian PAD has a narrow selectivity for the arginine containing protein material—not all proteins containing arginine residues could be modified using the mammalian PAD2 enzyme. It was shown that chicken egg white ovomucoid which contains an arginine residue at one of the trypsin inhibitory reactive site could not be modified by the mammalian PAD enzyme. In contrast, we show in here that a microbial PAD can decrease the trypsin inhibitory activity in chicken egg white ovomucoid making this enzyme suitable to be used in a wider variety of protein foods containing protease inhibitory activity. Also, prior studies have shown that the trypsin-chymotrypsin inhibitor, Bill, in peanut can be modified by a mammalian PAD enzyme, however not all arginine residues involved in the protease inhibitory activity can be modified (Tomofumi Kurokawa et al, J. Biochem, 1987, vol 101, p 1361-1367). This studies however looked at enzyme effect on purified peanut Bill protease inhibitor from peanut. In this invention it is shown that the microbial PAD can reduce trypsin inhibitory activity in the whole peanut, comprising more than just Bill trypsin inhibitory activities. The effect of the microbial enzyme PAD in this invention is seen as well in a more complex food such as processed peanut butter.

Surprisingly, the inventors of the present invention show that different aspects of sensory and/or mouthfeel and/or digestibility and/or protease inhibitory activity of a protein comprising food can be improved using PAD.

In one of its aspects, the invention relates to a process for modifying sweetness, liquorice, astringency, powdery/chalk, fulness, thickness and/or digestibility and/or protease inhibitory activity of a protein comprising food comprising

incubating a protein solution with a peptidyl arginine deiminase (PAD) and

optionally processing said PAD-treated protein solution into a protein comprising food.

Preferably, the process of the invention modifies at least one of the mentioned properties, i.e. a process of the invention provides a method for modifying (preferably decreasing) sweetness of a protein comprising food. Alternatively, the invention provides a process for modifying (preferably decreasing) liquorice of protein comprising food. The invention also provides a process for modifying (preferably decreasing) astringency of a protein comprising food. The invention further provides a process for modifying (preferably decreasing) powdery/chalk of a protein comprising food. Alternatively, the invention provides a process for modifying (preferably decreasing) fulness of a protein comprising food. The invention also provides a process for modifying (preferably decreasing) thickness of a protein comprising food. The invention further provides a process for modifying (preferably increasing) digestibility of a protein comprising food. The invention also provides a process for modifying (preferably decreasing) protease inhibitory activity of a protein comprising food. In another preferred embodiment, the process of the invention modifies at least 2, 3 or 4 of the mentioned properties, such as a process for modifying powdery/chalk, fulness and thickness of a protein comprising food.

The term “sweetness” as used herein refers to the basic taste most commonly perceived when eating foods rich in sugar.

The term “liquorice” as used herein refers to a taste sensation/component present in the sweet root of Glycyrrhiza glabra or in stevia plant species.

The term “astringency” as used herein refers to the dry, puckering mouthfeel similar to that caused by tannins that are for example found in many fruits or by proteins, especially in an acidic environment.

The term “powdery/chalk” as used herein refers to the degree in which the product feels powdery or grainy in the mouth.

The term “fulness” as used herein refers to the feeling of full, round body in the mouth.

The term “thickness” as used herein refers to firmness in the mouth and represents the force needed to push the product in between tongue and palate

The term “digestibility” as used herein refers to the capability of being digested and relates to the quantity of food that is retained by the body versus the quantity of food that is consumed. Digestibility of a protein directly relates to the extent of hydrolysis that digestive proteases such as pepsin and pancreatic peptidases, obtained upon incubation with the said protein under physiological relevant conditions.

The term “protease inhibitory activity” as used herein refers to an activity (preferably a protein) that inhibits the function of proteases (enzymes that aid the breakdown of proteins).

The term “modifying” refers to either increasing or decreasing depending on the aimed goal. For example, the invention provides a process for increasing the digestibility of a protein comprising food comprising

incubating a protein solution with a peptidyl arginine deiminase (PAD) and

optionally processing said PAD-treated protein solution into a protein comprising food.

The invention also provides a process for decreasing sweetness, liquorice, astringency, powdery/chalk, fulness and/or thickness of a protein comprising food comprising

incubating a protein solution with a peptidyl arginine deiminase (PAD) and

optionally processing said PAD-treated protein solution into a protein comprising food.

The invention also provides a process for decreasing protease inhibitory activity of a protein comprising food comprising

incubating a protein solution with a peptidyl arginine deiminase (PAD) and

optionally processing said PAD-treated protein solution into a protein comprising food.

Whether or not any of the mentioned properties is increased or decreased is determined by comparing to an otherwise identical prepared food except that the protein solution is not incubated with PAD.

The term “protein comprising food” refers to a food which comprises protein as one its components or as an added ingredient, i.e. a food comprising a protein. The protein comprising food preferably comprises at least 0.1% protein. Any suitable protein may be used in a process of the present invention. Advantageously the protein comprises protein bound arginine, such as at least 1 mol %, 2, 3, 4, 5 or at least 6 mol %.

The protein solution (alternatively: the food protein ingredient) is a liquid protein composition comprising a protein. In case the digestibility of a protein comprising food is modified (preferably increased) the protein solution comprises a protease inhibitor activity. In case the protease inhibitory activity is modified (preferably decreased) the protein solution comprises a protease inhibitor activity. As used herein, the term “protease inhibitor activity” is a peptide which added to a protease will decrease the protease activity. The term “protein solution” also includes a solution in which not all components are dissolved, i.e. the term “protein solution” also includes a protein suspension.

The step of “incubating a protein solution with a peptidyl arginine deiminase (PAD)” can be performed at any suitable pH for any suitable time and with any suitable enzyme concentration. The skilled person is very well capable of establishing a suitable enzyme amount or a suitable incubation temperature or a suitable incubation pH or a suitable incubation time, for instance incubating protein with a peptidyl arginine deiminase at a pH of between 4 and 9, such as a pH of between 5 and 8.5, such as a pH of between 5.5 and 8, such as a pH between 6 and 7, or a pH of between 6.2 and 6.8, for instance at a pH of about 6.5. A suitable temperature at which protein is incubated with PAD may be between 20 and 60 degrees Celsius, such as a between 30 and 50, or between 35 and 45 degrees Celsius.

The PAD-treated protein solution can for example be a final product, such as—but not limited to—a protein drink. Alternatively, the PAD-treated protein solution is a food ingredient which can subsequently be further processed into a protein comprising food.

As described above, any suitable protein can be used in a process of the invention. In a preferred aspect, the protein in a process of the invention is a plant or vegetable protein (the terms are used interchangeably herein). Preferably, a protein solution as used in a process of the invention is a solution which comprises a plant protein such as soy protein, rapeseed protein, wheat protein, buckwheat protein, maize protein, barley protein, potato protein, rice protein, oats protein, pea protein, (pea)nut protein, lupin protein, almond protein or said protein is egg protein, milk protein, gelatine protein or microbial protein.

A process of the invention comprises at least 1 step of incubating a protein solution with a peptidyl arginine deiminase (PAD). As described above and depending on whether the protein solution is a food ingredient or a final protein comprising food, a process of the invention optionally comprises the step of processing said PAD-treated protein solution into a protein comprising food. Depending on the starting material a further optional can be included in a process of the invention. In case the starting material is a flour such as—but not limited to—a plant protein flour, a process of the invention will include a step of dissolving flour to obtain a protein solution. Dissolving of a flour typically involves the addition of a liquid (for example water or a buffer) to the flour and allowing the flour to at least partly dissolve in said liquid. Depending on the characteristics of the flour it might be needed to mix the flour with the liquid for a certain amount of time optionally some heat to improve/speed up the dissolving. Alternatively, a suspension is prepared in water or a suitable buffer. I.e. an optional additional step of a method of the invention is: comprising dissolving flour to obtain a protein solution or preparing a suspension from flour.

The term protein arginine deiminase and peptidyl arginine deiminase (PAD) are used interchangeably herein. Protein or peptidyl arginine deiminases belong to a family of enzymes (EC 3.5.3.15) which convert peptide or protein bound arginine into peptide or protein bound citrulline. This process is called deamination or citrullination. In the reaction from arginine to citrulline, one of the terminal nitrogen atoms of the arginine side chain is replaced by an oxygen. The reaction uses one water molecule and yields ammonia as a side product (http://en.wikipedia.org/wiki/Citrullination). Whereas arginine is positively charged at a neutral pH, citrulline is uncharged. Surprisingly, it was found that a protein wherein at least part of the arginine has been converted into citrulline, and thereby resulting in protein with less charge, exhibited modified sweetness, liquorice, astringency, powdery/chalk, fulness, thickness and/or digestibility.

Peptidyl arginine deiminase (PAD) may be derived from any suitable origin, for instance from mammalian or microbial origin. PAD's used in the present invention are advantageously derived from a microbial source, i.e. the PAD used in a process of the invention is a microbial PAD. For instance, PAD's may be derived from fungal origin such as from Fusarium sp. such as Fusarium graminearum, Chaetomium globosum, Phaesphaeria nodorum or from bacterial origin such as from the bacteria Streptomyces, eg Streptomyces scabies, Streptomyces clavuligeres. The wording “derived” or “derivable” from with respect to the origin of a polypeptide as disclosed herein, means that when carrying out a BLAST search with a polypeptide as disclosed herein, the polypeptide may be derivable from a natural source, such as a microbial cell, of which an endogenous polypeptide shows the highest percentage homology or identity with the polypeptide as disclosed herein.

Peptidyl arginine deiminases are for instance known from WO2008/000714. WO2008/000714 discloses a process for enzymatically treating a protein with a protein arginine deiminase, wherein at least 30% of the arginine is transformed into citrulline.

A peptidyl arginine deiminase may be a pure or purified peptidyl arginine deiminase. A pure of purified peptidyl arginine deiminase is an enzyme that may be at least 50% pure, e.g., at least 60% pure, at least 70% pure, at least 75% pure, at least 80% pure, at least 85% pure, at least 80% pure, at least 90% pure, or at least 95% pure, 96%, 97%, 98%, 99%, 99.5%, 99.9% pure for instance as determined by SDS-PAGE or any other analytical method suitable for this purpose and known to the person skilled in the art.

Preferably, the used peptidyl arginine deiminase is Ca²⁺-independent. More preferably, the used peptidyl arginine deiminase is a microbial PAD and is Ca²⁺-independent.

Advantageously, peptidyl arginine deiminase as used in a process of the invention is a polypeptide which has at least 80%, 85%, 90%, 95%, 96%, 97%, 98%, 99% or 100% identity to the amino acid sequence of SEQ ID NO: 1, or to the mature amino acid sequence of SEQ ID NO: 1, wherein the polypeptide has peptidyl arginine deiminase activity.

For the purpose of this invention, it is defined here that in order to determine the percentage of sequence identity of two amino acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more amino acids. The sequence identity is the percentage of identical matches between the two sequences over the reported aligned region. The percent sequence identity between two amino acid sequences may be determined using the Needleman and Wunsch algorithm for the alignment of two sequences. (Needleman, S. B. and Wunsch, C. D. (1970) J. Mol. Biol. 48, 443-453). Both amino acid sequences and nucleotide sequences can be aligned by the algorithm. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, P. Longden, I. and Bleasby, A. Trends in Genetics 16, (6) pp 276-277, http://emboss.bioinformatics.nl/). For protein sequences EBLOSUM62 is used for the substitution matrix. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

A “mature polypeptide” is defined herein as a polypeptide in its final form and is obtained after translation of a mRNA into a polypeptide and post-translational modifications of said polypeptide. Post-translational modifications include N-terminal processing, C-terminal truncation, glycosylation, phosphorylation and removal of leader sequences such as signal peptides, propeptides and/or prepropeptides by cleavage.

A mature polypeptide sequence of SEQ ID NO: 1 may comprise or contain amino acids 19, 20, 21, 22, 23, 24 to 640 of the amino acid sequence of SEQ ID NO: 1, advantageously the mature polypeptide sequence of SEQ ID NO: 1 comprises or contains amino acids 22 to 640 of SEQ ID NO: 1, wherein methionine at position 1 in SEQ ID NO: 1 is counted as number 1.

The term “polypeptide” refers to a molecule comprising amino acid residues linked by peptide bonds and containing more than five amino acid residues. The term “protein” as used herein is synonymous with the term “polypeptide” and may also refer to two or more polypeptides. Thus, the terms “protein” and “polypeptide” can be used interchangeably. Polypeptides may optionally be modified (e.g., glycosylated, phosphorylated, acylated, farnesylated, prenylated, sulfonated, and the like) to add functionality. Polypeptides exhibiting activity in the presence of a specific substrate under certain conditions may be referred to as enzymes.

A peptidyl arginine deiminase, or polypeptide having peptidyl arginine deiminase activity may be produced in any suitable host organism by known methods in the art, for instance in fungi Aspergilli, eg Aspergillus niger or Aspergillus oryzae, Trichoderma, or the yeasts Saccharomyces, and Kluyveromyces or the bacteria of the genus Streptomyces or Bacilli. A suitable method to express a polypeptide having peptidyl arginine deiminase activity in Aspergillus niger is for instance disclosed in Examples 3 and 4 in WO2008/000714, which is herein included by reference.

The term “protein comprising food” as used herein refers to any type of food as long as the food at least comprises a protein. The term food refers to solid foods as well as drink.

In one of its embodiments, the invention provides a process for modifying sweetness, liquorice, astringency, powdery/chalk, fulness, thickness and/or digestibility and/or protease inhibitory activity of a protein comprising food comprising
incubating a protein solution with a peptidyl arginine deiminase (PAD) and
optionally processing said PAD-treated protein solution into a protein comprising food, wherein said protein comprising food is a protein comprising drink, i.e. a liquid intended for consumption.

The protein comprising drink can be any type of drink but in a preferred aspect, the protein comprising drink is a plant protein drink or a fermented plant protein product. Examples of suitable plant protein drinks are soy bean drink, pea drink, peanut drink, barley drink, rice drink, oat drink, quinoa drink, almond drink, cashew drink, coconut drink, hazelnut drink, hemp drink, sesame seed drink, wheat drink, potato drink or sunflower seed drink. Examples of a suitable fermented plant protein product are fermented soy bean product, fermented pea product, fermented peanut product, fermented barley product, fermented rice product, fermented oat product, fermented quinoa product, fermented almond product, fermented cashew product, fermented coconut product, fermented hazelnut product, fermented hemp product, fermented sesame seed drink, fermented wheat product, fermented potato product or fermented sunflower seed product.

Soy bean protein is commonly used to prepare an infant, follow-on or toddler drink. Properties like sweetness, liquorice, astringency, powdery/chalk, fulness, thickness and/or digestibility can be improved by using a process of the invention. The invention therefore provides, a process for modifying sweetness, liquorice, astringency, powdery/chalk, fulness, thickness and/or digestibility and/or protease inhibitory activity of a protein comprising food comprising

incubating a protein solution with a peptidyl arginine deiminase (PAD) and

optionally processing said PAD-treated protein solution into a protein comprising food, wherein said protein comprising food is a protein comprising drink, wherein said drink is infant, follow-on or toddler drink and wherein said protein is soy bean protein.

The produced protein comprising drink can be drink which produced without or with additional protein. Protein fortified (plant) protein drinks provide advantages such as improved nutritional value but are typically conceived as being difficult to consume due to undesired properties such as powdery/chalk, fulness, thickness. Protein fortified (plant) protein drinks prepared with a process of the invention have improved (reduced) powdery/chalk, fulness, thickness and hence the invention provides a process for modifying sweetness, liquorice, astringency, powdery/chalk, fulness, thickness and/or digestibility and/or protease inhibitory activity of a protein comprising food comprising

incubating a protein solution with a peptidyl arginine deiminase (PAD) and

optionally processing said PAD-treated protein solution into a protein comprising food, wherein said protein comprising food is a protein fortified (plant) protein drink. An examples of a protein fortified drink is a protein fortified drink for elderly or (post-surgery) recovering people to increase their caloric intake. Alternatively, such a protein fortified drink is a medicinal or clinical drink or a sport drink.

A protein comprising drink prepared by a process of the invention can have a neutral or an acidic pH.

The invention further provides use of a peptidyl arginine deiminase (PAD) for modifying sweetness, liquorice, astringency, powdery/chalk, fulness, thickness and/or digestibility and/or protease inhibitory activity of a protein comprising food. Preferably, the invention provides use of a peptidyl arginine deiminase (PAD) for modifying liquorice, astringency, powdery/chalk, fulness, thickness and/or digestibility and/or protease inhibitory activity of a protein comprising food. Preferably, the invention provides use of a peptidyl arginine deiminase (PAD) for modifying liquorice, powdery/chalk, fulness, thickness and/or digestibility and/or protease inhibitory activity of a protein comprising food. Preferably, the invention provides use of a peptidyl arginine deiminase (PAD) for modifying powdery/chalk, fulness, thickness and/or digestibility and/or protease inhibitory activity of a protein comprising food. The above described features of a process of the invention are equally applicable to the use part.

The directly obtained product from any of the herein described processes are also within the scope of present invention.

In another aspect, the invention provides a nutraceutical composition comprising a peptidyl arginine deiminase (PAD), preferably a microbial peptidyl arginine deiminase (PAD). More preferably, the microbial peptidyl arginine deiminase (PAD) has at least 80% identity to SEQ ID NO:1, or has at least 80% identity to the mature amino acid sequence of SEQ ID NO:1.

The term nutraceutical as used herein denotes the usefulness in both the nutritional and pharmaceutical field of application. Thus, the novel nutraceutical compositions can find use as supplement to food and beverages, and as pharmaceutical formulations or medicaments for enteral or parenteral application which may be solid formulations such as capsules or tablets, or liquid formulations, such as solutions or suspensions. As will be evident from the foregoing, the term nutraceutical composition also comprises food and drinks as obtained by any of the herein described methods as well as supplement compositions, for example dietary supplements.

The term dietary supplement as used herein denotes a product taken by mouth that contains a “dietary ingredient” intended to supplement the diet. The “dietary ingredients” in these products may include: vitamins, minerals, herbs or other botanicals, amino acids, and substances such as enzymes, organ tissues, glandules, and metabolites. Dietary supplements can also be extracts or concentrates, and may be found in many forms such as tablets, capsules, softgels, gelcaps, liquids, or powders. They can also be in other forms, such as a bar, but if they are, information on the label of the dietary supplement will in general not represent the product as a conventional food or a sole item of a meal or diet.

The invention will be explained in more detail in the following example, which are not limiting the invention.

### EXPERIMENTAL PART

Materials

Cloning and Expression of Peptidyl Arginine Deiminase

Cloning and expression of the polypeptide having peptidyl arginine deiminase activity according to SEQ ID NO: 1, was performed as disclosed in Examples 3 and 4 of WO2008/000714.

Peptidyl Arginine Deiminase (PAD) Activity

Peptidylarginine deiminase activity was determined by measuring the formation of citrulline residues in α-N-Benzoyl-L-arginine-ethyl ester (BAEE). The incubation mixture contained 100 mM tris-HCl buffer (pH 7.5), 5 mM CaCl₂), 10 mM DTT, 10 mM BAEE in a final volume of 700 μl. Incubation was performed at 55° C. for 30 min, and the reaction was stopped by adding 100 μl 8 N HClO₄. Citrulline was determined by colorimetry according the method of Guthöhrlein and Knappe, (1968) Anal. Biochem. 26, 188.

One unit of peptidyl arginine deiminase is expressed as 1 μmol of citrulline formed/min/mg of protein.

### Example 1

Reduction of Trypsin Inhibitory Activity (TIA) in Proteins Treated with PAD Enzyme

Various proteins containing TIA were incubated with PAD and the reduction in the TIA was measured with two different assays. The preparation of the proteins with and without PAD incubation and the assays used to measure TIA are described below.

Soy drink sample preparation: 400 μl Commercial soy drink (Provamel unsweetened) from the local store was diluted with 600 μl tap water. Subsequently 0.17 U/ml PAD was added and the solutions were incubated for 2.5 hours at 40° C. (Thermomixer 600 rpm). The control sample was incubated with the same volume of water instead of PAD.

Soy flour sample preparation: One g soy was mixed with 49 g 0.01 N NaOH. The pH of this suspension was adapted to 10 with 4 N HCl. The suspension was mixed thoroughly with magnetic stirring for 3 hours at ambient temperature. Subsequently the sample was centrifuged for 10 minutes at 3200*g. The supernatant (without the lipid containing upper part was centrifuged again for 10 minutes at 20817 g. To 150 μl of the resulting supernatant 0.04 U PAD was added and the solution was incubated for 30 minutes at 45° C. (Thermomixer 600 rpm). The control sample was incubated with the same volume of water instead of PAD.

Chicken egg white ovomucoid sample preparation: ovomucoid (Sigma) was solubilized (0.25 mg/ml) in 100 mM HEPES, 5 mM CaCl₂, 5 mM DTT, pH 7.2. To this, 0.17 U/ml PAD was added and incubated at 37° C. The reaction was stopped with 25 μl 0.2 M EDTA.

Rapeseed Cake Sample Preparation:

To 100 mg of rapeseed cake (obtained from cold-pressed rapeseed oil seed meal) 900 μl 2% NaCl was added and 0.17 units of PAD. The rapeseed was solubilized for 30 minutes at 55° C. in a Thermomixer at 800 rpm. Hereafter the sample was centrifuged for 10 minutes at 20817 g. The control rapeseed cake sample was solubilized in the absence of PAD.

Rapeseed Protein Isolate (RPI) Sample Preparation:

The RPI was prepared as described in the patent WO 2018/007492 starting from cold-pressed rapeseed oil seed meal. A 2% (w/v) solution of RPI was prepared from this sample. Subsequently 0.17 U/ml PAD was added and the solution was incubated for 2.5 hours at 40° C. (Thermomixer 600 rpm). The control sample was incubated with the same volume of water instead of PAD.

Peanuts and peanut butter sample preparation: Peanuts (in shell) were bought from a local store. The shell was removed and one part of peanuts were mixed with four parts of water (w/w). Peanut butter was purchased at a local store (Terra Sana brand) and mixed with 3 parts of water (w/w). To 500 μl of this suspension 0.4 U PAD or water (control) was added and incubated for 2 h at 40° C. Subsequently 1 part was diluted with 2 parts 10 mM acetic acid. 125 μl was analyzed as described below (TIA measurements).

Barley Sample Preparation

One gram of barley was mixed (magnetic stirring) in 10 ml water. To 500 μl of this suspension 0.4 U PAD was added and incubated for 90 minutes at 45° C. (Thermomixer 600 rpm). As a control water was added instead of PAD.

Buckwheat Flour and Buckwheat Drink Sample Preparation:

A 10% solution of buckwheat flour bought from the local store was prepared in water with mixing. The biological buckwheat drink bought from the local store (brand name Isola) was used as such. To 500 μl of this suspension 0.4 U PAD was added and incubated for 100 minutes at 45° C. (Thermomixer 600 rpm). For the control incubation, water was added instead of PAD. The trypsin inhibitory activity was measured as described below using the AzoCasein assay.

Lupin Sample Preparation

Lupin seeds were received from a local producer and were further processed into a 10% flour suspension in water. To 500 μl of this suspension 0.4 U PAD was added and incubated for 100 minutes at 45° C. (Thermomixer 600 rpm). For the control incubation, water was added instead of PAD. The trypsin inhibitory activity was measured as described below using the AzoCasein assay

TIA Measurements

TIA Measurement with BAEE Assay

The assay used in here is essentially as described in the Sigma protocol “assay method for trypsin inhibitor activity” (https://www.sigmaaldrich.com/technical-documents/protocols/biology/enzymatic-assay-of-trypsin-inhibitor.html). 25 μl 1 mg/ml Trypsin in 1 mM HCl (Sigma; Trypsin from bovine pancreas; 15267 units/mg solid) was added to 1.875 ml 1 mM HCl. Subsequently 100 μl of the protein sample prepared as described above (with or without enzyme incubation) was added into the reaction mixture. As a control, 100 μl 1 mM HCl was added instead of the inhibitor in the trypsin activity measurement. These solutions were incubated for 5 minutes at ambient temperature.

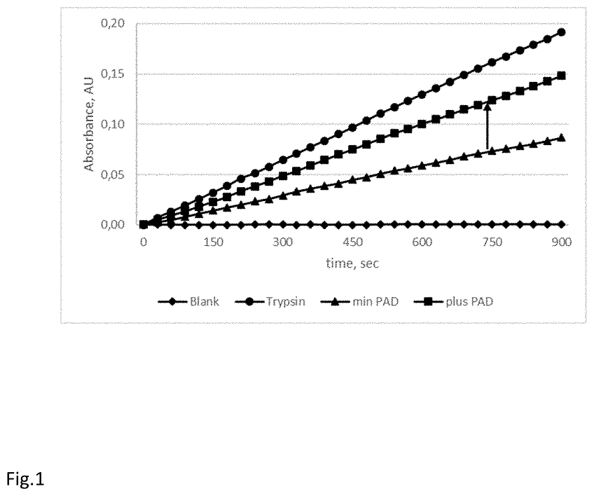

To 2.7 ml 67 mM sodium phosphate pH 7.6 (25° C.) 300 μl BAEE (Sigma; Na-Benzoyl-L-Arginine ethyl ester hydrochloride 0.86 mg/ml in the same buffer) was added and 100 μl 1 mM HCl. Trypsin activity was determined after addition of 100 μl of the Trypsin solutions and the reaction was monitored at 253 nm. The Spectrophotometer was calibrated with a blank containing 100 μl 1 mM HCl instead of the trypsin solution. The reaction was followed for 20 minutes with 3 data points per minute (FIG. 1).

An increase in absorbance at 253 nm (the slope of the line increased) indicates cleavage of the BAEE by the trypsin. When trypsin is incubated with the protein sample prior the substrate BAEE addition, the absorbance at 253 nm is decreased in time which indicates inhibition of the trypsin enzyme activity. When protein sample is incubated with PAD prior addition to the trypsin, the absorbance at 253 nm is (partially) restored indicating that trypsin inhibition by the protein sample is diminished. From the slopes with and without PAD treated samples a reduction of TIA levels by PAD can be calculated. Trypsin inhibition (%) was calculated by: 100−(sample-sample background)/(Trypsin-blank).

TIA Measurement Using Azo Casein Assay

The TIA levels in protein materials were assessed using the assay described in: “Quantitative Determination of Trypsin Inhibitory Activity in Complex Matrices”, Robin E. J. Spelbrink et al.; The Open Food Science Journal, 2011, 5, 42-46. In brief, 125 μl protein sample (or 125 μl 10 mM acetic acid for blank) was mixed with 25 μl 0.35 mg/ml Trypsin in 1 mM hydrochloric acid. The reaction was started by the addition of 30 mg/ml Azocasein in 100 mM Tris, 5 mM calcium chloride at pH 8.5. For background signals Trypsin was replaced by 1 mM HCl. After 30 minutes at 37° C. the samples were quenched with 150 μl 15% TCA. Non-hydrolyzed protein material and other insoluble were removed by centrifugation for 10 minutes at 15000*g at 4° C. 100 μl was transferred into a 96 well microtiter plate and mixed with 100 μl 1.5 M NaOH. The absorbance at 450 nm was measured. Trypsin inhibition (%) was calculated by: 100−(sample-sample background)/(Trypsin-blank).

In the Table 1, the reduction of TIA measured by the BAEE or Azocasein assay for the proteins described above is presented:

TABLE 1
reduction of TIA in proteins treated with PAD

| | TIA reduction with PAD |
|---|---|
| Protein source | treatment, % |
| Soy drink^{a} | 33 |
| Chicken ovomucoid^{a} | 25 |
| Rapeseed cake^{a} | 88 |
| Rapeseed protein | 71 |
| isolate (RPI)^{a} | |
| Buckwheat flour^{b} | 71 |
| Buckwheat drink^{b} | 46 |
| Peanut^{b} | 78 |
| Peanut butter^{b} | 76 |
| Barley seeds^{b} | 90 |
| Lupin seeds^{b} | 61 |

^{a}TIA measured using BAEE assay;
^{b}TIA measured using AzoCasein assay

### Example 2

TIA Quantification in Soy Drink and Soy Flour Using International Standard Method

The international standard method for measuring trypsin inhibitory activity was used by Eurolin labco (EN-ISO 14902:2001) to measure the trypsin inhibitory activity of soy flour and soy drink before and after enzymatic treatment with PAD.

Enzyme Treatment of Soy Drink and Soy Flour:

Soy drink (Provamel unsweetened) bought from the local store: 300 g was incubated (magnetic stirring) in a water-bath for 90 minutes at 45° C. with 0.04 U PAD/g soy drink. As a control soy drink was incubated without any addition. Subsequently the samples were stored overnight at −20° C. Hereafter the samples were freeze dried.

Soy Flour: a 10% suspension (w/w) of raw soy flour was prepared in a solution of 0.01 M NaOH. The pH was adjusted to pH 10. Subsequently the suspension was mixed for 3 hours at ambient temperature (magnetic stirring). Hereafter the suspension was divided into two equal parts (200 g) and moved to a water-bath at 45° C. After 15 minutes to one part 0.05 U PAD/g soy flour suspension was added and incubated for 30 minutes. Subsequently the samples were stored overnight at −20° C. Hereafter the samples were freeze dried.

The results of the TIA measurements are shown in Table 2 below.

TABLE 2
Quantification of TIA levels in soy drink and soy
flour before and after PAD enzyme incubation.

| | TIA, | reduction TIA, % |
|---|---|---|
| Sample type | mg/g prot. | with PAD treatment |
| Soy drink | 13 | |
| Soy drink + PAD | 8.5 | 35 |
| Soy flour | 28.6 | |
| Soy flour + PAD | 16.3 | 43 |

### Example 3

Improved Digestibility of Rapeseed Protein Using In Vitro Model

Rapeseed protein sample treatment with PAD: rapeseed protein isolate (RPI) prepared as described in patent WO 2018/007492. To 500 ml 10% RPI in water 43 U PAD was added. This suspension was incubated for 3 hours at 40-45° C. Subsequently the sample was frozen and lyophilized.

The in vitro digestibility was measured at NIZO Food Research Center using the SYMPHYD platform as described in He, Tao & Giuseppin, Marco. (2013). Slow and fast dietary proteins differentially modulate postprandial metabolism. International journal of food sciences and nutrition. 65. 10.3109/09637486.2013.866639.

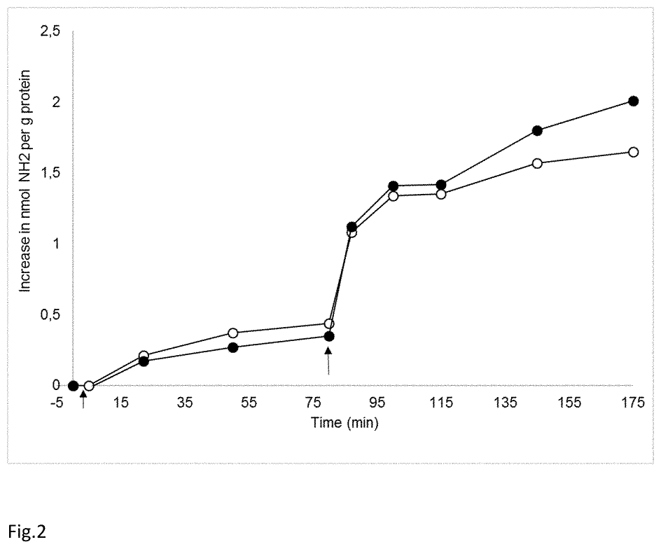

The results of the in vitro model for protein digestion of RPI and RPI treated with PAD are shown in FIG. 2.

An increase in ammonia released (measured by OPA method) indicates a higher digestibility of the protein sample. More than 20% increase in digestibility is observed at the end of digestion for the RPI treated with PAD compared to untreated sample.

### Example 4

Improved Mouthfeel of Soy Drink Treated with PAD

Incubation of Soy Drink with PAD

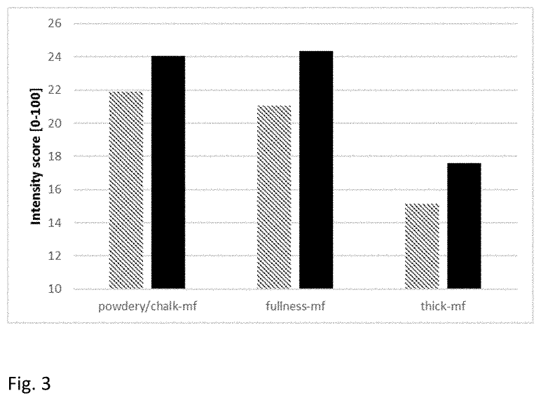

Soy drink Provamel unsweetened: 1000 ml (4*250 ml) was incubated in a water-bath (shaking 40 rpm) for 4 hours at 45° C. with 0.27 U/ml U PAD. As a control soy drink was incubated without any addition. Subsequently the samples were heated with the microwave to 65° C. and kept at that temperature for 5 minutes to inactivate the PAD enzyme. Hereafter the samples were cooled in ice-water. A sensory panel assessment was then performed on these samples. The samples were evaluated by means of quantitative descriptive analysis (QDA) as described in Meilgaard M., Civille G V., Carr B T. 2007. Sensory Evaluation Techniques. 4th ed. Boca Raton, Fla. CRC Press., with a panel (n=12) on attributes relevant for the products in the test. During the test the samples were offered according to an optimally balanced design and were scored on 0-100 unstructured line scales in EyeQuestion in duplicate. The data were analyzed with an ANOVA to find significant differences between the individual samples. Differences with p<0.05 were considered as significant. The results of the sensory panel assessment are shown in FIG. 3. A significant decrease in the mouth feel was observed regarding attributes such as powdery, fulness and thickness in the mouth for the enzyme treated drinks.

### Example 5

Sensory Assessment of Rapeseed Protein Treated with PAD Enzyme

Per sample, 1000 ml 2% protein powder suspensions were made (plant protein powders corrected for protein content) in tap water and pH adjusted to pH −6.5 with 4M H₂SO₄.

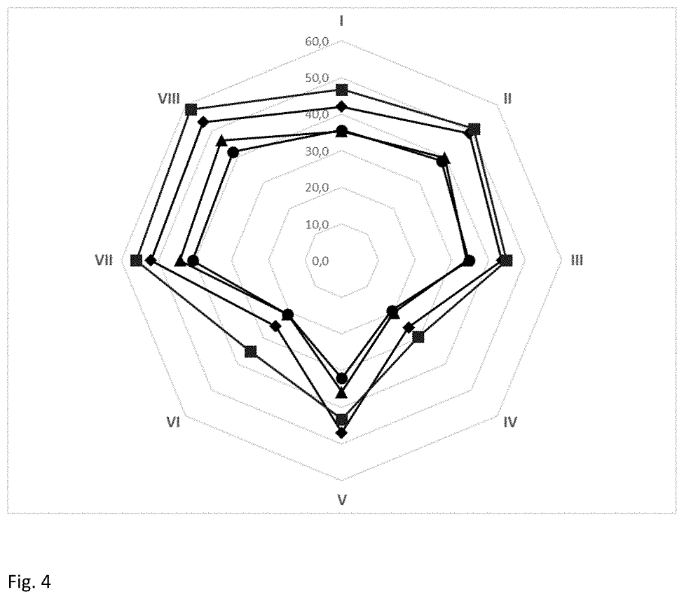

Suspensions were incubated for 2 h at 45° C. with and without PAD enzyme addition at different enzyme dosage. The enzyme was inactivated, by heating the materials at 65° C., with a holding time of 5 minutes. The samples were evaluated by means of descriptive analysis (QDA) with the sensory panel (n=13) on attributes relevant for the product in the test. During the test the samples were offered according to an optimally balanced design and were scored on 0-100 unstructured line scales in EyeQuestion in duplicate. The data were analyzed with an ANOVA to find significant differences between the individual samples. Differences with p<0.05 were considered as significant (FIG. 4). By treatment of RPI with PAD enzyme, all sensory attributes such as sweetness, liquorice, astringency, bitter, length aftertaste (as described in the figure legend), tested were decreased with increasing the amount of PAD added.

### Example 6

IEF Decrease in Pea and Soy Proteins Drinks Treated with PAD

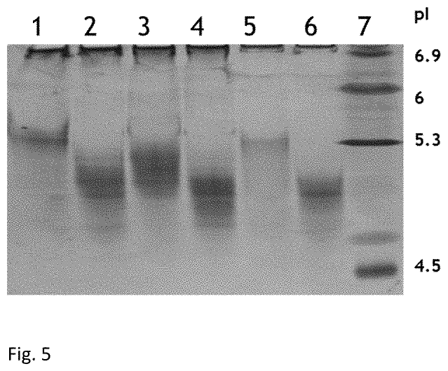

Samples were diluted four times with H₂O. Subsequently the samples were diluted 1 to 1 with IEF sample Buffer. 10 μl of the samples were applied to the Novex™ pH 3-7 IEF Protein Gel. Total run time 2.5 hours (according to Invitrogen protocol). The reduction in the pl for soy drink and pea drinks after PAD treatment is shown in FIG. 5. The reduction of pl of these proteins is consistent with PAD enzyme activity on these proteins which reduces the number of positive charges on the proteins. A similar reduction in the isoelectric point of almond proteins was measured in the almond flour treated with PAD (data not shown). The shift of the pl for these proteins to more acidic values allow a better solubility of these proteins in neutral beverage formulations.

### Example 7

Stability of PAD Enzyme at Acidic pH

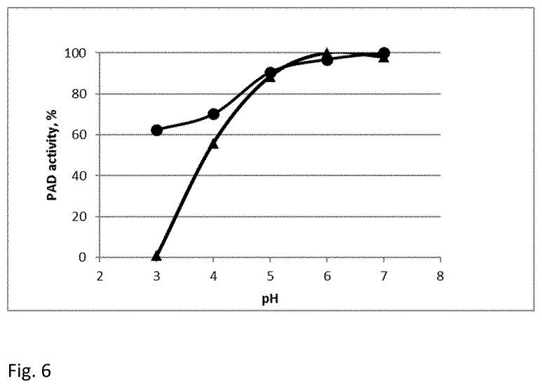

Stability of the PAD enzyme was evaluated after 40 minutes incubation at two temperatures (on ice at 4° C. and in a water bath at 37° C.) in a pH range from 3 to 7. For the pH 3 to 5, the PAD sample was diluted 6 times in 100 mM citric acid buffer and the pH was raised by addition of 1N sodium hydroxide. For the pH above 5 the enzyme was diluted in 50 mM potassium phosphate buffer. The enzyme activity was measured as described in WO2017/009100A1. FIG. 6 shows the results of this experiment. PAD enzyme has a maximum activity at pH 7 in this experiment, however at pH 4 about 50% of activity remains at the enzyme incubation for 40 min at 37° C. showing that the enzyme can be active in conditions similar to those found in the stomach of mammals.

1. Process for modifying sweetness, liquorice, astringency, powdery/chalk, fulness, thickness and/or digestibility and/or protease inhibitory activity of a protein comprising food, said process comprising
incubating a protein solution with a peptidyl arginine deiminase (PAD) and
optionally processing said PAD-treated protein solution into a protein comprising food.

2. The Process according to claim 1, wherein said protein solution is a food ingredient.

3. The Process according to claim 1, wherein said protein is a plant protein optionally soy protein, rapeseed protein, wheat protein, buckwheat protein, maize protein, barley protein, potato protein, rice protein, oats protein, pea protein, (pea)nut protein, lupin protein, almond protein or wherein said protein is egg protein, milk protein, gelatine protein or microbial protein.

4. The Process according to claim 1, further comprising dissolving flour to obtain a protein solution.

5. The Process according to claim 1, wherein said PAD is a microbial PAD.

6. The Process according to claim 1, wherein the peptidyl arginine deiminase (PAD) has at least 80% identity to SEQ ID NO:1, or has at least 80% identity to the mature amino acid sequence of SEQ ID NO:1.

7. The Process according to claim 1, wherein said protein comprising food is a protein comprising drink.

8. The Process according to claim 7, wherein said protein comprising drink is a plant protein milk or a fermented plant protein product.

9. The Process according to claim 7, wherein said plant protein drink is soy bean drink, pea drink, peanut drink, barley drink, rice drink, oat drink, quinoa drink, almond drink, cashew drink, coconut drink, hazelnut drink, hemp drink, sesame seed drink, wheat drink, potato drink or sunflower seed drink.

10. The Process according to claim 9, wherein said drink is infant, follow-on or toddler drink and wherein said protein is soy bean protein.

11. The Process according to claim 7, wherein said (plant) protein drink is a protein fortified (plant) protein drink.

12. The Process according to claim 7, wherein said (plant) protein drink is a drink with neutral or acidic pH.

13. The Process according to claim 7, wherein said (plant) protein drink is a medicinal drink or a sport drink.

14. A product comprising a peptidyl arginine deiminase (PAD) for modifying liquorice, astringency, powdery/chalk, fulness, thickness and/or digestibility of a protein comprising food.

15. A nutraceutical composition comprising a peptidyl arginine deiminase (PAD), optionally a microbial peptidyl arginine deiminase (PAD).

16. A nutraceutical composition according to claim 15, wherein the microbial peptidyl arginine deiminase (PAD) has at least 80% identity to SEQ ID NO:1, or has at least 80% identity to the mature amino acid sequence of SEQ ID NO:1.
